(19) United States
(12) Patent Application Publication
(10) Pub. No.: US 2026/0218098 A1
(43) Pub. Date: Jul. 30, 2026

(54) SYSTEM FOR CONTROLLING A THREE-DIMENSIONAL PRINTING PROCESS

(71) Applicant: Biomotion Technologies FlexCo, Vienna (AT)

(72) Inventors: Gregor WEISGRAB, Vienna (AT); Diego CASTANEDA-GARAY, Vienna (AT)

(73) Assignee: Biomotion Technologies FlexCo, Vienna (AT)

(21) Appl. No.: 19/146,771

(22) PCT Filed: Jan. 10, 2023
(86) PCT No.: PCT/EP2023/050481

§ 371 Date: Jul. 9, 2025

(51) Int. Cl. C12M 3/00; B33Y 10/00; B33Y 50/02

(52) U.S. Cl. CPC C12M 21/08; B33Y 10/00; B33Y 50/02

(57) ABSTRACT

The invention relates to a system for controlling a three-dimensional printing process. The system comprises a sensing signal providing unit (101) configured to provide a sensing signal indicative of a printed material property at an evaluation location. The evaluation location is a printing location satisfying a predefined relationship with respect to a current printing location (10) and can, in particular, correspond to the current printing location. The presented system further comprises a mismatch determining unit (102) configured to determine a mismatch between the printed material property and a printed material target property for the evaluation location based on the sensing signal, and a control parameter determining unit (103) configured to determine a control parameter for the three-dimensional printing process based on the determined mismatch. The system allows for an increased reproducibility of three-dimensional printing results.

### FIELD OF THE INVENTION

The invention relates to a system, a method and a computer program for controlling a three-dimensional printing process. Furthermore, the invention relates to an apparatus for three-dimensional printing.

### BACKGROUND OF THE INVENTION

It has been observed that results of three-dimensional printing processes can vary significantly, thereby potentially leading to relatively large variations among printed objects, even if identical results are aimed at. The causes for such unintentional variations in three-dimensional printing results can be difficult to control. One such cause can be inhomogeneities in a printing material used, for instance, which are essentially random.

In the article “In situ process monitoring and automated multi-parameter evaluation using optical coherence tomography during extrusion-based bioprinting” by Yang et al., Additive Manufacturing, volume 47 (2021), a protocol for scanning a printed object like a three-dimensional scaffold using an optical coherence tomography (OCT) system whose probe is mounted next to and oriented parallel to an extrusion nozzle in a longitudinal direction is described. According to the scanning protocol, laterally overlapping single OCT datasets are acquired, each covering a lateral area of 10 mm by 10 mm, wherein these datasets are subsequently laterally combined to image layers. It is proposed to determine such an image layer for each printed layer of the object, i.e. after the layer is finished, and to use the corresponding OCT data for determining a surface profile of the printed layer or parameters related to a filament or pore size in the printed layer. This allows for a monitoring of the printing process to some degree. However, since the complete image layers are constructed only after the printing of the respective layer has been finished, wherein only then the layers are analyzed, printing inaccuracies that have occurred in a layer can no longer be corrected. Hence, it can remain difficult to reproduce final printing results.

### SUMMARY OF THE INVENTION

It is an object of the invention to allow for an increased reproducibility of three-dimensional printing results.

In a first aspect of the present invention, a system for controlling a three-dimensional printing process is presented. The system comprises a) a sensing signal providing unit configured to provide a sensing signal indicative of a printed material property at an evaluation location, the evaluation location being a printing location satisfying a predefined relationship with respect to a current printing location, b) a mismatch determining unit configured to determine a mismatch between the printed material property and a printed material target property for the evaluation location based on the sensing signal, and c) a control parameter determining unit configured to determine a control parameter for the three-dimensional printing process based on the determined mismatch.

Since a sensing signal indicative of a printed material property at an evaluation location is provided, wherein the evaluation location is a printing location satisfying a predefined relationship with respect to a current printing location, local information about printing results during an ongoing printing process becomes accessible, which allows for a local, and if desired real-time, monitoring of the ongoing printing process. Since a mismatch between the printed material property and a printed material target property for the evaluation location is determined based on the sensing signal, wherein a control parameter for the three-dimensional printing process is determined based on the determined mismatch, the local information about the printing results in the ongoing printing process can be used to control the printing process more accurately, thereby allowing for an increased reproducibility of any final printing results.

Preferably, the evaluation location corresponds to the current printing location. In other words, the sensing signal providing unit is preferably configured to provide a sensing signal indicative of a printed material property at an evaluation location corresponding to the current printing location. That the evaluation location “corresponds” to the current printing location is to be understood herein such that the evaluation location coincides with the current printing location up to an uncertainty with which the current printing location and the location from which the sensing signal is acquired can be determined and/or controlled. That the evaluation location corresponds to the current printing location means that the predefined relationship between the evaluation location and the current printing location is a correspondence between the two locations. In other words, the predefined relationship satisfied by the evaluation location with respect to the current printing location can be such that the evaluation location is a printing location corresponding to the current printing location.

While it may be preferred that the evaluation location corresponds to the current printing location, this is not necessary. For instance, it may be preferred that the evaluation location corresponds to a printing location behind the current printing location, such as, for instance, to a printing location 1 to 5 mm behind the current printing location. A printing location “behind” the current printing location can be understood as a location preceding the current printing location along a printing path or, more generally, in a printjob. A printjob may be defined as a set, particularly a sequence, of commands required to conduct a production process. Further embodiments in which no sensing signal is provided that would be indicative of a printed material property at an evaluation location corresponding to the current printing location will be apparent from the subsequent description. Evaluation locations different from the current printing location may be beneficial for evaluating a final state of the printed material. Viscous printing materials, for instance, have been observed to still change their properties after deposition for some time. The evaluation location may therefore optionally be chosen such that only printed material is evaluated that has already settled, i.e. which has been printed a predetermined settling time before. The settling time may be predefined, such as based on previous printing processes, for instance.

In fact, the printing process may be controlled differently all together, i.e. without any sensing signals, without any determined mismatches and/or without any determined control parameters. The printing process may not even be a process where anything is printed. Conversely, any of the data processing described herein may be used not to control a process, but instead, for instance, a method or apparatus. The measures described herein are also not necessarily used for any control.

Preferably, the sensing signal providing unit is configured to provide the sensing signal while printing is in progress. This allows for a “live” monitoring of the printing process. Optionally, also the mismatch determining unit is configured to determine the mismatch between the printed material property and the printed material target property for the evaluation location while printing is in progress. In this way, potentially necessary adaptations to the printing process can be taken into account earlier. In particular, the control parameter determining unit can be configured to determine the control parameter for the printing process, which is determined based on the determined mismatch, while printing is in progress.

In fact, the evaluation location and/or the predefined relation between the evaluation location and the current printing location can be chosen depending on the printing process, such as depending on a type of the printing process. For instance, the system can comprise a planned printing path data providing unit configured to provide planned printing path data, the planned printing path data indicating whether a planned printing path along which printing is to be carried out proceeds in layers, wherein the sensing signal providing unit can be configured to choose the evaluation location such that, if the planned printing path data indicate that the planned printing path proceeds in layers, the evaluation location and the current printing location lie within a same layer. If desired, printing inaccuracies can in this way be timely corrected, without having to wait until printing a subsequent layer. Printing variations across different layers can hence be minimised.

A printing process based on a printing path proceeding in horizontal layers corresponding to different height levels could also be regarded as a printing process involving a continuous movement of the current printing location at a plurality of distinct heights. However, in principle, layered printing is not limited to horizontal layers. Instead, the layers could also be oriented differently in space. Conversely, printing processes involving a continuous movement of the printing location at a plurality of distinct heights are not limited to layered printing. Instead, the current printing location can move in space without forming any layers.

If the printing process does involve a continuous movement of the current printing location at a plurality of distinct heights, the predefined relationship satisfied by the evaluation location with respect to the current printing location can be predefined such that the evaluation location is at the same height as the current printing location. In this way, printing inaccuracies can, if desired, be corrected without having to wait until printing at a subsequent height, such that printing variations across different heights can be minimized.

A height could also be referred to as a location in a vertical direction. The vertical direction is not limited to a direction of, for instance, a gravitational force, but could be defined with respect to any reference system used to describe the printing process. If, for instance, the printing process involves a deposition of material along a longitudinal direction at continuously changing lateral locations, the height could be measured along the longitudinal direction. Moreover, the plurality of distinct heights can correspond to predefined height differences. For instance, they may define equidistant height levels, which could be referred to as layers. Accordingly, in layered printing, i.e., when the printing process involves printing layer by layer, the predefined relationship satisfied by the evaluation location with respect to the current printing location could be predefined such that the evaluation location lies within a layer currently being printed.

The three-dimensionality of the printing process to be controlled preferably refers to a spatial three-dimensionality. That is to say, the printing process preferably refers to a printing at several different positions in space. The controlled printing process could also be one- or two-dimensional, i.e. in space. Hence, in principle, the printed objects could be line-like, planar or volumetric objects.

That the sensing signal is indicative of the printed material property at the evaluation location can be understood such that the sensing signal carries information based on which at least one value of the printed material property at the evaluation location can be determined or estimated. This may particularly be the case if the sensing signal has been acquired from, or at, the evaluation location, such as by interaction with printed material at the evaluation location. However, also sensing signals acquired from, or at, printing locations away from the evaluation location may still carry sufficient information about the printed material target property at the evaluation location. Depending on, for instance, a rate of change of the printed material target property along a path of printed material, a sensing signal acquired from a printing location away from the evaluation location may still be used for determining, at least to some approximation, a value of the printed material property at the evaluation location.

In preferred embodiments, the units of the system are configured to carry out their respective functions continuously, thereby implementing a continuous feedback loop, wherein each cycle of the loop is associated with a different one of a plurality of subsequent printing locations. Accordingly, it may be preferred that the evaluation location is the current printing location. In other words, the predefined relationship between the evaluation location and the current printing location may particularly be an equality, at least to a reasonable approximation. The feedback loop may particularly be a control feedback loop, meaning that the control parameter may be indicative of a printing parameter for the current printing location as well, or at least for an immediately following printing location. The immediately following printing location may be a printing location temporally following the current printing location in the ongoing printing process by an amount that may be predefined or implied by a time needed to determine the control parameter.

While the control parameter may be indicative of a printing parameter for the current printing location, this is not necessary. In particular, the control parameter can also be indicative of a printing parameter for a printing location following the current printing location in the ongoing printing process by a significant and/or intentional amount. Conversely, if the control parameter is indicative of a printing parameter for the current printing location, the evaluation location may be a printing location preceding the current printing location in the ongoing printing process by a significant and/or intentional amount. The sensing signal provided by the sensing signal providing unit may, for instance, have been acquired in the past, such as at a time when a respective current printing location coincided with the evaluation location. More generally, and particularly irrespective of which printing location the control parameter is determined for, the evaluation location may be different, i.e. significantly and/or intentionally different, from the current printing location. Hence, the predefined relationship between the evaluation location and the current printing location may indicate a spatial relation between the evaluation location and the current printing location, particularly a spatial distance along a printing path of the printing process. Optionally, the spatial relation is such that the evaluation location and the printing location are within a predefined maximum distance from each other. For instance, when printing in layers, i.e. when printing an object by subsequently printing layers of the object, it may be preferred that the evaluation location and the current printing location are within a same layer, wherein the control parameter may then particularly be indicative of a printing parameter for the current printing location.

The mismatch determining unit is not necessarily configured to determine a mismatch between the printed material property and the printed material target property. In particular, even if the quantity determined by the mismatch determining unit is determined based on the printed material property and the printed material target property, the determined quantity is not necessarily a mismatch per se. More generally, the mismatch determining unit may be configured to determine a mismatch indicator based on the printed material property and the printed material target property, the mismatch indicator being indicative of a mismatch between the printed material property and the printed material target property. For instance, the mismatch indicator may be a function of the printed material property and the printed material target property, wherein a value of the function indicates a mismatch between the printed material property and the printed material target property. Hence, the mismatch determining unit may also be referred to as a mismatch indicator determining unit.

In an embodiment, if a mismatch indicator indicating a mismatch between the printed material property and a printed material target property is determined for the current printing location, the control parameter determining unit may be configured to assess whether an immediate change in a control parameter can decrease the mismatch, and to determine a control parameter indicative of a printing parameter for a later, i.e. not immediately following, printing location otherwise. In layer-based printing, for instance, the later printing location may correspond to the current printing location in the two dimensions parallel to the layers, i.e. only differ in the dimension perpendicular to the layers. However, as also indicated further below, the determined control parameter can also relate to one or more later printing location distributed across the subsequent layer, thereby potentially indicating adapted printing schemes layer-by-layer.

In fact, a plurality of evaluation locations can be used. That is to say, the sensing signal providing unit may be configured to provide one or more signals indicative of the printed material property at a plurality of evaluation locations, each of the evaluation locations being a printing location satisfying a respective predefined relationship with respect to the current printing location. The mismatch determining unit can be configured to determine a mismatch indicator based on mismatches between the printed material property and the printed material target property determined for each of the evaluation locations, or based on indicators thereof. The control parameter determining unit may be configured to determine the control parameter for the three-dimensional printing process based on the determined mismatch indicator. In this way, the acquired local information about the printing results during the ongoing printing process can be combined to evaluate the printing results in more extended regions without sacrificing an evaluation accuracy. The plurality of evaluation locations can define an evaluation region of printed material, wherein the evaluation region may refer, for instance, to a section of a line, or strand, of printed material whose ends correspond to two different printing times. In some embodiments, the evaluation region as a whole can be chosen to precede a current printing location by a predefined amount, wherein the control parameter may be determined for the current printing location. In this way, a region-wise printing can be implemented, wherein printing of any given region of an object can be controlled based on a mismatch, or indicator thereof, between a printed material property and a printed material target property determined for a previously printed region.

While multiple evaluation locations may be considered for determining the control parameter, also one or more control parameters can be determined. For instance, a plurality of control parameters may be determined based on a mismatch, or indicator thereof, between the printed material property and the printed material target property determined only for a single evaluation location. Likewise, the plurality of control parameters may be determined based on a mismatch, or indicator thereof, between the printed material property and the printed material target property determined for a plurality of evaluation locations, i.e. based on sensing signals provided for the plurality of evaluation locations. The plurality of control parameters may be indicative of printing parameters at a plurality of printing locations. For instance, each control parameter, which may be determined based on mismatches, or indicators thereof, determined for one or for a plurality of evaluation locations, may be indicative of a printing parameter for a respective different printing location.

If, for instance, the printing process is layer-based, based on a mismatch, or indicator thereof, determined in a layer currently being printed, one or more control parameters may be determined indicating an adapted printing pattern, i.e. new toolpaths, for a subsequent layer to be printed.

The printing process can be a bioprinting process. Bioprinting refers to printing processes in which biological materials are used as printing material, particularly printing processes in which biological materials are deposited. Exemplary biological materials used for bioprinting are cells and collagens. Besides the biological material being printed, also nonbiological materials like non-biological polymers, for instance, can be deposited in bioprinting processes. The printing result of a bioprinting process can be biological tissue. Depositing non-biological, or “non-living”, materials in addition to biological materials can mechanically reinforce the printed tissue. In an embodiment, the bioprinting process involves depositing bioink or biomaterial inks as defined in the article “A definition of bioinks and their distinction from biomaterial inks” by J. Groll et al., Biofabrication, volume 11 (2019), which is herewith incorporated by reference in its entirety.

Irrespective of the kind of printing material used, the term “printing”, as used herein, can be understood so as to include any kind of additive manufacturing. Depending on the particular printing process, the physical mechanism underlying the “printing” may be described more accurately by different terms. Nevertheless, all of them will be collectively referred to herein as “printing”. Thus, for instance, “jetting” as executable with devices like an inkjet is understood as a particular kind of printing. Likewise, without being limited thereto, “printing” is understood herein to include any kind of material deposition, such as a deposition of printing material onto a print bed and/or onto previously deposited printing material. Thus, while the three-dimensional printing process to be controlled can, for instance, be extrusion-based, i.e. based on an extrusion of printing material, a same or similar control can be applied to printing processes which are not extrusion-based. The printing process to be controlled can be a process which itself does not include any material deposition. For instance, previously provided material, which may possibly initially still be liquid, can, in a subsequent processing step, be hardened by directed, or patterned, radiation. Accordingly, also lithographic and tomographic printing techniques may be controlled as described herein. The previous providing of printing material may or may not be carried out in a manner that would be referred to as material deposition. Irrespective of this, if viewed on its own, the subsequent processing, i.e. the hardening of the material, will normally not include any material deposition and could therefore be understood as a non-deposition type of printing process. A “hardening” of material may refer to a solidification, particularly a crosslinking, of material.

Thus, “printing” locations are not limited to material deposition locations, but can generally refer to locations where printing, i.e. any kind of printing, takes place. For instance, a printing location can be a location where printing material, which may previously have been deposited in at least partially liquid form, is being hardened, in which case the printing location may be referred to as a hardening location. The hardening may be temporally decoupled from any previous providing, particularly depositing, of printing material, i.e. does not necessarily immediately follow. For instance, one or more layers of printing material or even all material for an object to be printed may be deposited before active material hardening is initiated. On the other hand, it is also possible that material deposition takes place while active material hardening is carried out, at a current deposition location and/or a past deposition location.

Accordingly, a printing path is not to be misunderstood as being limited to a deposition path, such as a toolpath followed by a tool, like an extruder, used for material deposition. Instead, the printing path may generally refer to a sequence of printing locations followed during a printing process. As indicated above, the printing locations may also refer to hardening locations. Material hardening, particularly if carried out by radiation, can be effected at a plurality of locations simultaneously. In fact, also material deposition can, in principle, be carried out at a plurality of locations simultaneously. Hence, the printing path does not necessarily correspond to a single curve in space, but could also refer to a plurality of curves in space or even a temporal sequence of printing regions. For example, in lithographic printing processes, entire layers can be cross-linked simultaneously, and in tomographic printing processes even entire three-dimensional objects. This can be achieved by irradiation with spatially extended light patterns. As already indicated further above, a printing path in this more general sense may also be understood as corresponding to, being part of, or implied by a printjob.

In particular, the printing process can be any one or any combination of the following: extrusion-based, inkjet-based, laser-assisted, stereo-lithographic, based on two-photon polymerization, based on melt-electrospinning writing, based on electrohydrodynamic jetting, based on microfluidic deposition.

The printed material property can refer to any of the following: a location or alignment of the printed material, particularly with respect to printed material at a previous printing location, a shape parameter, a surface area, a porosity, a cellular density, a material and/or cell percentage, a material type distribution, a pore size, a strut size, a pore shape parameter, a pore interconnectivity, a degree of fusion between layers, a weight, a degree of humidity, a degree of cross-linking.

A cell is understood herein as a structural and/or functional unit of the printed material, particularly of an objected formed by the printed material. A pore is defined herein as referring to a spacing between lines of printed material, or more generally as a void between deposited material. A line of printed, or deposited, material can be understood herein as a strand or strut.

The degree of humidity can particularly refer to a humidity percentage. Likewise, the degree of cross-linking can particularly refer to a cross-linking percentage. Cross-linking can refer to the process of methacrylate groups in the printed material bonding together covalently when exposed to ultraviolet (UV) light, but could also include other forms of cross-linking, which may be enzymatically-based, based on click-chemistry (e.g. thiol-based), covalently-based, etc. For example, if some of the printed material is not cross-linked to a sufficient degree during the printing process, this can be adjusted afterwards. In this way, a more consistent stiffness of printed objects like printed tissue structures can be achieved, since polymers in the printed objects may be interlinked to a more uniform degree.

A degree of fusion between layers may, in the case of non-layer-based printing, refer to a degree of fusion between sections of printed material, such as strands, that have been printed on top of each other. The degree of fusion may be measured in terms of an overlap of printed material from the two sections, or layers. The overlap may be determined in a direction in which the printed material sections, or layers, lie on top of each other, which would often be referred to as a z direction. The degree of fusion between sections of printed material printed on top of each other, particularly between layers, is considered a good indicator for mutual adhesion and therefore mechanical stiffness of a printed object.

The sensing signal can be an imaging signal. The imaging signal can refer to any signal that could be used for imaging the printed material at the evaluation location. Hence, the imaging signal may be viewed as carrying imaging data. The imaging signal may be any of the following: an optical coherence tomography signal, a three-dimensional line scanner signal, an ultrasound signal, a multiphoton microscopy signal, a second harmonic generation signal, a confocal microscopy signal, a photoacoustic microscopy signal, a hyperspectral imaging signal, a Raman spectroscopy signal, a Lidar signal. Ultrasound sensing signals may be particularly preferred in case of submerged printing. Imaging signals in general, and particularly the mentioned types, allow for a very accurate evaluation of the printed material, since known and sophisticated image analysis techniques can be employed. In particular, geometric information about the printed material becomes naturally accessible. On the other hand, it may be preferred to not use a whole image, but only the imaging signal. The time needed for reconstructing an image from the imaging signal may in this way be saved, while possibly still being able to accurately control the printing process. For instance, the mismatch determining unit may be configured to determine mismatches, or indicators thereof, between printed material properties and printed material target properties already based on spectrograms provided by the sensing signal providing unit, and not based on full images, even though full images could be determined based on the spectrograms.

An optical coherence tomography (OCT) system may be used to acquire the OCT signal. The OCT system may be a time domain OCT system, a swept-source OCT system, a spectral-domain OCT system, a full-field OCT system, a fibre-optical OCT system, a freespace OCT system, and variants of such technology, e.g., Doppler OCT, polarization OCT, optical coherence phase microscopy (OCPM), etc. Exemplary commercial OCT systems that could be used include, e.g., the Telesto series and GANYMEDE-II series of Thorlabs Corp., IVS-1000/2000 of Santec Corp., etc.

Technologies that could be used to acquire the multiphoton microscopy (MPM) signal include, for instance, two-photon fluorescence microscopy imaging technology, three-photon fluorescence microscopy imaging technology, multi-focal-point multiphoton microscopy technology or commercial multiphoton fluorescence microscopy imaging technology, e.g., the FVMPE-RS system of Olympus Corp., Bergamo-II series of Thorlabs Corp., etc.

The confocal microscopy (CM) signal may be acquired using a laser scanning confocal spectral imaging system, a spinning-disk confocal microscope system, a programmable array microscope system or a commercial CM system like, e.g., Zeiss LSM800 of Zeiss Corp., LEXT-OLS4100 of Olympus Corp., etc.

Hyperspectral imaging signals may be acquired as disclosed, for instance, in the article “Applications of hyperspectral imaging in the detection and diagnosis of solid tumors” by Zhang et al., Translational Cancer Research, volume 9 (2020), which is herewith incorporated in its entirety by reference.

Raman spectroscopy signals, which could also be referred to as Raman microscopy or just Raman imaging signals, may be acquired as disclosed, for instance, in the article “Non-Invasive Three-Dimensional Cell Analysis in Bioinks by Raman Imaging” by Marzi et al., ACS Applied Materials & Interfaces, volume 14 (2022), which is herewith incorporated in its entirety by reference.

Lidar signals, i.e. “light detection and ranging” or “light imaging, detection and ranging” signals, can be acquired with any of the known Lidar sensing devices, for instance.

The imaging signal may in fact be an imaging signal acquired using any microscope or any camera, including regular cameras.

The mismatch determining unit can be configured to determine the mismatch, or an indicator thereof, between the printed material property and the printed material target property for the evaluation location based on an image determined based on the imaging signal. Hence, one or more imaging signals used as sensing signals can be provided, wherein an image is constructed based on the one or more imaging signals, and wherein the mismatch, or the indicator thereof, is determined based on the image. The image may be an image, such as a cross-sectional image, of the printed material at the evaluation location. Moreover, the image will preferably correspond in type to the imaging signal based on which it is determined, i.e. it can particularly be any of the following: an OCT image, a three-dimensional line scan, an ultrasound image, a multiphoton microscopy image, a second harmonic image, a confocal image, a photoacoustic image, a hyperspectral image, a Raman image, a Lidar image.

While imaging signals may allow for a particularly accurate evaluation, the provided sensing signal could also be any of the following: a signal of a weight sensor for weighing an amount of deposited material, a signal of a pressure sensor for sensing a pressure inside an extruder used for printing, a signal of a force sensor for sensing a force indicative of how printing material is deposited (e.g., a force with which printing material is extruded in case of extrusion-based printing), a signal indicative of a current energy consumption of a motor used for extruding the printing material.

By sensing a pressure inside an extruder used for printing, blockages in the extruder may be detected. Certain pressures may also be measured using a force sensor. For instance, a force sensor in the form of a load cell may be configured to measure a back-pressure by being built into a syringe plunger used in case a syringe is used as extruder.

The weight sensor could, for instance, be placed below a print bed, such that it could measure the weight of the overall printed material. For instance, in extrusion-based printing, the measured weight would successively increase in the course of the printing process, wherein the increase could be compared to model increases. The model increases could also be referred to as target increases.

Non-imaging sensing signals, which can allow for a simpler and yet sufficiently accurate evaluation, may be provided by the sensing signal providing unit as an alternative or in addition to imaging signals. In the latter case, the sensing signals of the different types may be used in combination with the imaging signals for determining control parameters.

The printed material target property can be determined based on a printing model, wherein the printing model can be a model of an object to be printed. The modelled object, and hence the model, can be three-dimensional. Already a single-layered object may be considered three-dimensional. The model may be described in terms of G-code, but could also be described differently. For instance, in case of laser-based printing, the model may not be described in terms of G-code, but may be defined with respect to a (resonant or galvanometer) scanning system used for controlling the laser. Irrespective of the form in which it is provided, the model may be indicative of paths to be followed and/or movements to be carried out by a printing device during the printing process.

The printing model can be determined based on one or more previous printing processes. Hence, for instance, the printing model does not necessarily correspond to an originally desired printing result. Deviations in printing results from an originally desired printing result can be considered insignificant as long as actually achieved printing results are similar enough to each other. For instance, the printing model may be determined based on printing results of one or more previous printing processes by averaging or other statistical analysis.

The mismatch determining unit can be configured to determine the mismatch between the printed material property and the printed material target property for the current printing location, or the indicator thereof, based further on a predefined tolerance. In this way, the printing process can be controlled more efficiently, since unnecessary control loops, which would result in no or only insignificantly improved printing results can be avoided. For instance, mismatches below a predefined mismatch tolerance threshold may be ignored.

The control parameter can refer to any of the following: in case a) the printing process is extrusion-based, a material extrusion speed, a movement speed of an extruder used as printing device, a pneumatic pressure, a temperature, a retraction speed and/or length of the extruder, a distance of the extruder to a print bed and/or a temperature of the print bed, and in case b) the printing process is inkjet-based, a droplet size, an ejection speed, and/or an ejection frequency.

Generally, the control parameter may be indicative of any one or any combination of the following: a material deposition speed, a volume of printed material, a frequency, such as an open/closing frequency, a printhead movement, a laser intensity, a laser frequency, a movement speed. In the following an overview of printing parameters and/or characteristics possibly indicated by the control parameter for various printing techniques is given.

TABLE 1
Possible printing techniques and associated
printing parameters/characteristics

| | Exemplary printing parameters/ |
|---|---|
| Printing technique: | characteristics: |
| Printing using | Mixing ratio, speed, volume, material |
| microfluidic printheads | composition, coaxial ratio |
| Printing using | Back-pressure, valve open/closing |
| mircovalves | frequency, time of opening |
| Melt-Electrowriting (MEW) | Pressure, extrusion or flow rate, |
| | voltage, temperature |
| Cross-linking | Irradiation intensity, distance, |
| | exposure time |
| Coaxial | Mixing ratio, material ratio |
| Fused Filament | Extrusion characteristics like, e.g., |
| Fabrication (FFF) | an extrusion rate and/or back-pressure, |
| | retraction and/or other movement |
| | characteristics, temperature |
| Light/laser-based | Laser intensity, irradiation time, |
| | movement speed, wavelength, movement |
| | path |
| Electrohydrodynamic | Back-pressure, valve open/closing |
| Jetting | frequency, time of opening, pressure, |
| | extrusion or flow rate, voltage, |
| | temperature, surrounding humidity |

The control parameter determined by the control parameter determining unit may not just be indicative of, but can also be, i.e. directly correspond to, any of the mentioned printing parameters/characteristics. Regardless of the printing technique, there may always be machine parameters that can be determined, such as the movement speed and/or direction of a printing device. Such machine parameters may be considered control parameters in their own right.

The control parameter determining unit may be configured to determine more than one control parameter for the three-dimensional printing process based on the determined mismatch or mismatch indicator. It is understood that determining a respective control parameter may refer to determining a respective adapted control parameter, i.e. to adapting the respective control parameter. The control parameter determining may hence be configured to adapt one or more control parameters for the three-dimensional printing process. In fact, in view of the above mentioned desire to minimize unintentional variations in printed objects, the control parameter determining unit may be configured to adapt one or more control parameters such that one or more printed material properties remain constant. This may require holding some control parameters at a constant value. On the other hand, the control parameter determining unit may be configured to determine one or more control parameters without initial reference values, i.e. from scratch. This may be needed, for instance, when printing with a printing device and/or a printing material for the first time. Control parameters may then be found iteratively by starting with initial, potentially user-defined and/or estimated, control parameter values, printing, evaluating the printing result and adjusting the respective control parameters continuously during printing until a result of the evaluation is satisfactory, i.e., for instance, beyond a predetermined quality.

In particular embodiments, a) the sensing signal providing unit can be configured to provide sensing signals for each of a plurality of printing locations of an ongoing three-dimensional printing process, the, i.e. each of the, sensing signals being indicative of the printed material property at a respective one of the plurality of printing locations, i.e. itself, b) the mismatch determining unit can be configured to determine mismatches between the printed material property and the printed material target property for the plurality of printing locations, or indicators thereof, based on the respective sensing signals, and c) the control parameter determining unit can be configured to determine, based on a respective mismatch or mismatch indicator at a given one of the plurality of printing locations, the control parameter for a respective subsequent printing location.

The printing locations may differ significantly and/or intentionally from each other, or may immediately follow upon each other. In the latter case, as indicated further above, a distance between the printing locations may be predefined or implied by a time needed to determine the control parameter.

In a further aspect, the invention relates to an apparatus for three-dimensional printing, comprising a) a printing device for printing material in three dimensions, b) a sensing device for acquiring sensing signals indicative of a printed material property at a current printing location, and c) a controller for controlling the printing device based on a control parameter determined by the above system using the sensing signals acquired by the sensing device. It will be understood that, in variants in which the evaluation location does not correspond to the current printing location, the sensing device will be configured to acquire sensing signals indicative of the printed material property at the respective other evaluation location.

The controller may comprise the above system. Alternatively, the controller and the system may be separate devices communicating with each other, wherein the system may or may not be considered being included in the apparatus. In fact, also the units of the system, i.e. the system for controlling the three-dimensional printing process, are not necessarily part of one and the same physical device, but could be distributed over several devices.

The printing device and the sensing device can be movable relative to each other. In other words, the printing device may be movable relative to the sensing device and/or the sensing device may be movable relative to the printing device. Being “movable” preferably refers in this context to being movable via suitable control by the controller and corresponding actuation through, e.g., a motor. In particular, the sensing device may be movable so as to follow the printing device in a printing direction. For instance, an optical lens used for optical coherence tomography may be movable so as to be positioned behind the printing device in the printing direction throughout the printing process. Alternatively to imaging from behind, the lens may be movable so as to image the printing process from the top, from a side or from the bottom. The printing direction is preferably understood herein as the direction in which the printing location moves during printing.

Sensing signals can be acquired from, i.e. for, inner regions of the printed material, particularly when using optical coherence tomography. It will be understood, however, that a depth of the inner regions from, or for, which the sensing signals are acquired may be limited. Therefore, for instance, in layer-based printing, and when an optical lens used as sensing device is positioned so as to image the printing process from below, a printing height may be limited. When reaching a predefined limit printing height, the optical lens may then be repositioned, such as to the top or a side. This may allow to continue imaging the printing process with good quality.

It can be preferred that the sensing device and/or the printing device are movable such that the sensing device trails the respective current printing location over the whole printing process. In this way, sensing signals can always be acquired from “behind”, wherein from “behind” is to be understood relative to a current printing direction. To do so, the printhead (e.g. a syringe used for material deposition) may be centered, while a lens used as sensing device may be rotated around the printhead. Alternatively, instead of moving the lens itself, mirrors may be used for deflecting light towards the lens, in which case only the mirrors may be moved and the lens itself may be stationary. The mirrors may be positioned and/or moved such that imaging signals can be acquired through the lens as if the lens were trailing the printing device.

The sensing device can be configured such that the sensing signals are acquired from the evaluation location, particularly the current printing location itself, and/or from a region within a predefined distance from the current printing location. In particular, as indicated above, the sensing device can be rotatable relative to the printing device. The “rotatability” of the sensing device relative to the printing device preferably refers to a rotatability of the sensing device about, i.e. around, the printing device. However, additionally or alternatively, the sensing device may be rotatable about, i.e. around, an own axis. In particular, the controller may be configured to control a rotation of the sensing device relative to the printing device such that control signals can, if desired, be continuously acquired from the current printing location. In other words, the controller may be configured to control the rotation of the sensing device relative to the printing device such that a sensing aperture of the sensing device, through which sensing signals are acquired, faces the current printing location, particularly throughout the printing process, if desired.

Optionally, the apparatus comprises more than one printing device and/or more than one sensing device. All of the above may then apply to each combination of printing devices and sensing devices. In case of a plurality of printing devices, for instance, an equal number of current printing locations may be defined, each of them being associated with one of the printing devices. For each of the current printing locations, one or more evaluations may be defined, and each of the sensing devices may acquire sensing signals from evaluation locations associated with one or more current printing locations.

In a further aspect, the invention relates to a method for controlling a three-dimensional printing process, including a) providing a sensing signal indicative of a printed material property at an evaluation location, the evaluation location being a printing location satisfying a predefined relationship with respect to a current printing location, b) determining a mismatch between the printed material property and a printed material target property for the evaluation location, or an indicator thereof, based on the sensing signal, and c) determining a control parameter for the three-dimensional printing process based on the determined mismatch, or the indicator thereof.

Furthermore, a method for three-dimensional printing is presented, the method including a) printing material in three dimensions, b) acquiring sensing signals indicative of a printed material property at a current printing location, and c) controlling the printing device based on a control parameter determined according to the above method using the acquired sensing signals.

A further aspect of the invention relates to a computer program for controlling a three-dimensional printing process, the program comprising instructions causing a computer to carry out the above method for controlling a three-dimensional printing process.

Moreover, a computer program for three-dimensional printing is presented, the program comprising instructions which, when executed on a computer controlling the above apparatus for three-dimensional printing, cause the apparatus to execute the above method for three-dimensional printing. The computer may be included in, correspond to, or include the controller of the apparatus.

A further aspect of the invention relates to an object printed by the above apparatus for three-dimensional printing and/or according to the above method for three-dimensional printing.

It shall be understood that the system of claim 1, the apparatus of claim 14 and the method of claim 19 have similar and/or identical preferred embodiments, as defined in the dependent claims.

It shall be understood that a preferred embodiment of the invention can also be any combination of the dependent claims with the respective independent claim.

### BRIEF DESCRIPTION OF THE DRAWINGS

In the following drawings:

FIG. 1 shows schematically and exemplarily a system for controlling a three-dimensional printing process,

FIG. 2 shows schematically and exemplarily an apparatus for three-dimensional printing,

FIG. 3 shows schematically and exemplarily a further apparatus for three-dimensional printing,

FIG. 4A shows schematically and exemplarily a strand of printed material,

FIG. 4B shows schematically and exemplarily images of cross-sections of the strand of printed material, and

FIG. 5 shows schematically and exemplarily a method for controlling a three-dimensional printing process.

### DETAILED DESCRIPTION OF EMBODIMENTS

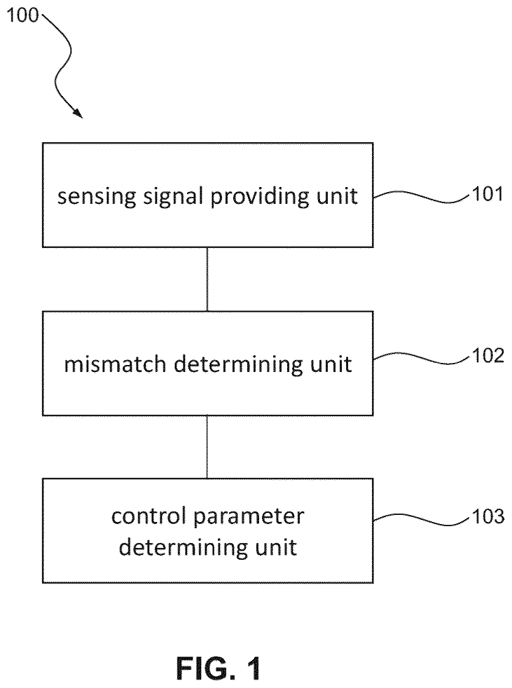

FIG. 1 shows schematically and exemplarily a system 100 for controlling a three-dimensional printing process. The system 100 comprises a sensing signal providing unit 101, which is configured to provide a sensing signal indicative of a printed material property at an evaluation location, wherein the evaluation location is a printing location satisfying a predefined relationship with respect to a current printing location. The predefined relationship can particularly be such that the evaluation location corresponds to the current printing location. Moreover, the sensing signal providing unit 101 is preferably configured to provide the sensing signal while printing is in progress.

The system 100 further comprises a mismatch determining unit 102 configured to determine a mismatch between the printed material property and a printed material target property for the evaluation location based on the sensing signal. For instance, the mismatch determining unit 102 may be configured to determine a difference between a) a value of the printed material property at the evaluation location based on the provided sensing signal and b) a target value of the printed material property for the evaluation location, wherein the target value may be one of several values of the printed material target property predefined for respective evaluation locations. In other embodiments, other mismatch indicators than differences may be chosen. For instance, any function monotonically decreasing or increasing with increasing mismatch, particularly difference, may alternatively be used as mismatch indicator.

The system 100 further comprises a control parameter determining unit 103 configured to determine a control parameter for the three-dimensional printing process based on the determined mismatch, i.e. the mismatch between the printed material property and the printed material target property determined by the mismatch determining unit 102 for the evaluation location. In particular, an adapted control parameter value can be determined, which may also be understood as adapting the control parameter. The determined control parameter, or the adaptation of the control parameter, is not necessarily carried out immediately, such as at the current printing location. Instead, the determined control parameter, or its adaptation, can refer to a further printing location. Moreover, it will be understood that, in the case that the sensing signal providing unit 101 is configured to provide the sensing signal while printing is in progress, the mismatch determining unit 102 and the control parameter determining unit 103 do not necessarily determine the mismatch and the control parameter, respectively, while printing is in progress as well. For instance, the mismatch and/or the control parameter may be determined while printing is paused, or even after the printing process has been finished, i.e. after an objected to be printed during the printing process has been completely printed. In the latter case, the determined control parameter may refer to a control parameter to be taken into account in a future printing process, such as for printing a further copy of a previously printed object. Nevertheless, for many applications, it may be preferred that the mismatch determining unit 102 and the control parameter determining unit 103 carry out their functions while printing is in progress.

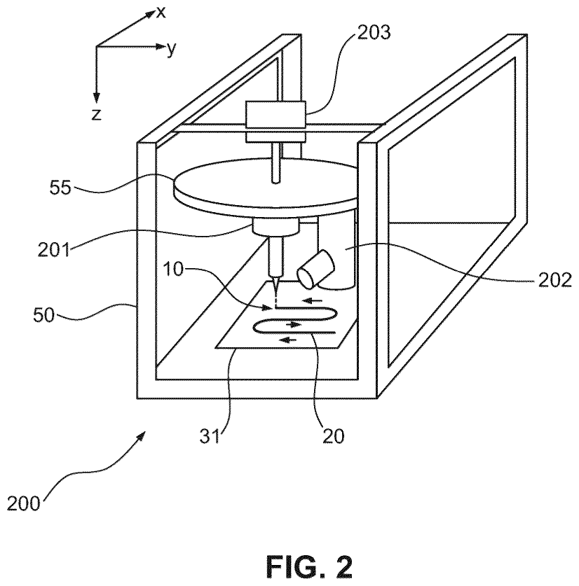

FIG. 2 shows schematically and exemplarily an apparatus 200 for three-dimensional printing. The apparatus 200 comprises a printing device 201 for printing material in three dimensions, a sensing device 202 for acquiring sensing signals indicative of a printed material property at a current printing location 10, and a controller 203 for controlling the printing device 101 based on a control parameter determined by the system 100 using the sensing signals acquired by the sensing device 202. The controller 203 may comprise the system 100, or may be structurally separate from, but communicating with the system 100.

The apparatus 200 shown in FIG. 2 comprises a frame 50 with a base on which a print bed 31 is positioned, and a vertical support structure to which the printing device 201 is mounted. In the example of FIG. 2, the printing device 201 is mounted to the vertical support structure of the frame 50 via a control device, which comprises the controller 203 and a motor controlled by the controller 203, and a disc 55, which is rotatable about a vertical axis (z axis). Apart from the rotation of the disc 55, the control device is moveable along the vertical support structure of the frame 50 in the two horizontal directions (x and y directions). While not specifically indicated in FIG. 2, the horizontal movement of the control device, and therefore also of the disc 55, can correspond to a movement of the control device along the part of the vertical support structure extending in y direction, and a movement of this part along the rest of the vertical support structure in x direction, wherein both of these movements, like the rotation of the disc 55, may be effected by the motor of the control device when being accordingly controlled by the controller 203 included in the control device. While also not being specifically indicated in FIG. 2, it will be understood that a power supply can be provided for the control device in an arbitrary manner.

Regarding the movement of the control device along the vertical support structure of the frame 50, the apparatus 200 could be viewed as an inverted x-y table. In fact, the apparatus 200 could, as far as the movement mechanism is concerned, in principle be inverted, in which case the base of the frame 50, including the print bed 31, could be moveable like an x-y table, while the control device could be fixed, such as fixed with respect to the vertical support structure. According to such an alternative arrangement, the same relative movement between the print bed 31 and the printing device 201 could be achieved as with the arrangement shown in FIG. 2.

Apart from being movable in the x-y plane, the printing device 201, and with it a respective current printing location 10, is movable along the z direction. For this purpose, for instance, the control device comprising the controller 203 and the motor may be configured to retract and/or protract the printing device 201 along the z direction. Additionally or alternatively, while not shown in FIG. 2, the control device may be movable in z direction relative to the frame 50, wherein this movement may translate, by virtue of the connection of the control device to the printing device 201, to a movement of the printing device 201 in z direction. In the above indicated alternative, “inverted” arrangement in which the control device, and with it the printing device 201 and the disc 55, would be fixed and instead the base of the frame 50, including the print bed 31, would be moveable, the latter, i.e. the base of the frame 50, could be moveable also in z direction. Hence, the x-y table could particularly be an x-y-z table.

Thus, relative movements of the printing device 201 with respect to the print bed 31, or vice versa, in all three spatial dimensions may be carried out. For instance, successive layers of printed material 20 may be printed on top of each other, each layer lying in an x-y plane at a different position in z direction. However, as already mentioned, three-dimensional objects may also be printed by proceeding differently than layer-by-layer, particularly differently than in successive x-y planes. For instance, parts of an object may first be printed completely in z direction, wherein only then printing may be continued for object parts at other positions in the x-y plane. Also, printing may be carried out on a print bed being tilted with respect to the x-y plane.

As exemplarily illustrated by FIG. 2, the printing device 201 and the sensing device 202 may be moveable relative to each other. Hence, not only may the printing device 201 and a surface on which printing takes place (e.g. the print bed 31) be moveable relative to each other, but additionally or alternatively a relative motion may be effectable between the printing device 201 and the sensing device 202. This also means that the sensing device 202 and the surface on which it is to be printed (e.g. the print bed 31) may be moveable with respect to each other. In the case of the exemplary apparatus 200 schematically illustrated by FIG. 2, the relative motion between the printing device 201 and the sensing device 202 is facilitated by means of the rotatable disc 55, since the sensing device 202 is mounted to the rotatable disc 55. More specifically, the printing device 201 is mounted to the disc 55 at the disc's centre, i.e. where the disc 55 is mounted to a rod connecting the disc 55 to the control device and serving as the rotational axis about which the disc 55 can rotate. The sensing device 202, on the other hand, is mounted to the disc 55 at a non-zero radial distance to the disc centre. In this way, if the disc 55 is controlled to rotate by the controller 203, the printing device 201, and therefore also the current printing location 10, does not change (as long as the disc is not otherwise horizontally moved), while the sensing device 202 is rotated about the printing device 201. Hence, as the printing direction changes in the course of the printing process, the current printing location 10 can be tracked from a constant viewing angle relative to the printing direction.

In the apparatus 200 shown in FIG. 2, the current printing location 10 is controlled in terms of translational movements in the x and/or the y and/or the z direction, while a relative motion between the printing location 10 and the sensing device 202 can be controlled via the rotation of the disc 55. In an alternative embodiment of the apparatus 200, the mounting positions of the printing device 201 and the sensing device 202 to the rotatable disc 55 could be switched. In that case, the current printing location 10 would result from a combination of translational movements in the x and/or the y and/or the z direction and rotations of the disc 55, while still the relative motion between the current printing location 10 and the sensing device 202 would be determined through the rotation of the disc 55.

Irrespective of where the printing device 201 and the sensing device 202 are mounted to the disc 55 of the apparatus 200, the sensing device 202 can be moved so as to follow the printing device 201 in the printing direction. In FIG. 2, a strand of printed material 20 is shown, wherein arrows indicate the printing direction which has continuously changed while printing the strand of printed material 20. The printing path followed according to FIG. 2 has a snakelike shape, but any other printing path, including non-connected paths, would be possible.

Moving the sensing device 202 so as to follow the printing device 201 in the printing direction preferably refers to a control of movements of the printing device 201 and the sensing device 202 by the controller 203 such that a spatial relation between the printing device 201 and the sensing device 202 with respect to the printing direction, i.e. the respective current printing direction, remains constant during the printing process. For instance, the spatial relation between the printing device 201 and the sensing device 202 with respect to the printing direction may be expressed in terms of an angle between a virtual line or plane defined by, particularly connecting, the printing device 201 and the sensing device 202, and the printing direction, wherein the controller 203 may control the movement of the printing device 201 and the sensing device 202 such that this angle remains constant. In the exemplary apparatus 200 shown in FIG. 2, for instance, the printing path is about to take a further turn to the right (as viewed in printing direction), which may be realized by a translational (combined x-y) movement of the disc 55 in the horizontal plane, wherein during this turn the disc 55 may be controlled to rotate clockwise (when viewed from above, i.e. in z direction) at an angular speed which is chosen in dependence on the mounting position of the sensing device 202 to the disc 55, the printing speed (i.e. the horizontal speed of the printing device 201) and the turning radius of the printing path such that the sensing device 202 can continuously acquire sensing signals from the advancing current printing location 10 at a constant angle with respect to the changing printing direction. In this way, in the hypothetical case of a perfectly printed strand of material 20 having a constant width and also uniform further structural properties, the printed material 20 at the current printing location would locally always “look” the same for the sensing device 202.

As indicated in FIG. 2, the sensing device 202 may be configured, i.e. accordingly arranged and also otherwise suitable, for acquiring sensing signals from the current printing location 10. For instance, the sensing device 202 may be oriented such that a sensing aperture of the sensing device 202 through which the sensing signals are acquired faces the current printing location. In the example of FIG. 2, the orientation of the sensing device 202 with respect to the rotatable disc 55 may be fixed. In embodiments in which the evaluation location does not correspond to the printing location 10, the sensing device 202 may be oriented such that its sensing aperture faces the respective evaluation location different from the printing location 10. Moreover, in embodiments in which sensing signals are acquired from several evaluation locations, including the printing location 10 or not, the sensing aperture of the sensing device 202 may be adapted accordingly, i.e. have a sufficiently broad sensing angle, for instance. In this way, sensing signals may be acquired from the evaluation location, which may be the current printing location or not, and predefined regions surrounding the evaluation location.

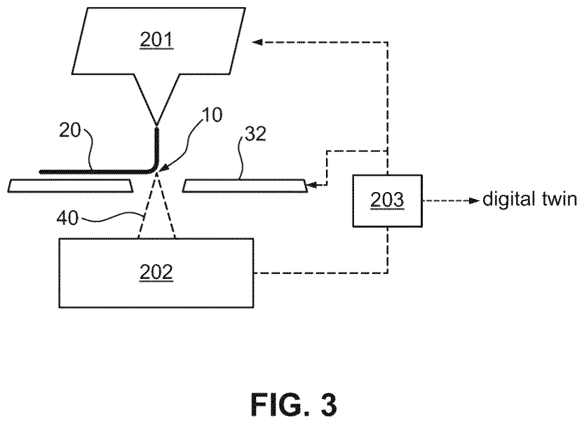

While, in the exemplary apparatus 200 shown in FIG. 2, the sensing device 202 is rotatable around the printing device 201 so as to stay, as viewed in printing direction, “behind” the current printing location 10 throughout that printing process, such a relative rotation between the printing device 201 and the sensing device 202 may not be necessary in other embodiments. In particular, any relative movement between the printing device 201 and the sensing device 202 is generally not limited to a rotational movement. Moreover, the sensing signals may be acquired from a different angle with respect to the printing direction. FIG. 3, for instance, illustrates a case where the sensing signals are acquired from “below” instead of from “behind”. Likewise, the sensing device 202 from FIG. 2 could be moved so as to view the current printing location not from “behind”, but from the side, such as at an angle of 90 degrees with respect to the current printing direction, for example. Generally, also more than one sensing device could be used, such that sensing signals from several angles can be acquired, wherein each of the sensing devices could be moved individually. It is also not necessary that sensing signals are acquired from a same angle throughout. For instance, a sensing device could be used to acquire first sensing signals from a first angle with respect to a currently printed strand of material at a first point in time, and to acquire second sensing signals from a second angle with respect to a currently printed strand of material at a second point in time, wherein the first and the second angle are different from each other and the relative motion between the printing device and the sensing device is adapted so as to allow for the changed angle of sensing signal acquisition between the first point in time and the second point in time. Hence, it is not necessary for the control of the printing process that the printed material always “looks the same” to any sensing device. In contrast, evaluating the printing process with different viewing angles may allow for acquiring yet more complete information about the printing process and therefore a yet more accurate control. Moreover, for printing processes which use inkjets as printing device or which are based on melt-electrospinning writing, it may not always be possible to keep a) the evaluation location with respect to the current printing location and/or b) the angle at which the sensing signals are acquired with respect to the current printing direction, i.e. an evaluation direction, fixed, since the current printing location and/or direction may change too quickly for the sensing device to follow.

FIG. 3 shows schematically and exemplarily an apparatus for three-dimensional printing according to a different embodiment than the one illustrated by FIG. 2. Nevertheless, the apparatus shown in FIG. 3 can, in principle, comprise the same printing device 201, the same sensing device 202 and the same controller 203 as the apparatus 200 shown in FIG. 2. Differences between the apparatus shown in FIG. 3 and the apparatus 200 shown in FIG. 2 may only lie in how the controller 203 controls movements of the printing device 201 and the sensing device 202, and in how these movements are realized, i.e. how the printing device 201 and the sensing device 202 are structurally mounted and mechanically driven. Moreover, in order to acquire sensing signals 40 from “below” as indicated in FIG. 3, a printing base 32 may be chosen to be transparent or comprise a hole for the sensing signals 40. The printing base may be the surface of an x-y-z table on which a transparent print bed (not shown in FIG. 3), possibly in the form a Petri dish, lies and on which printing is carried out. The region of transparency, or the hole, in the printing base may extend over the whole (transparent) print bed, such that sensing signals can be acquired from all potential printing locations. On the other hand, as indicated in FIG. 3, it is also possible that the printing base comprises one or more confined holes through which the sensing device 202 can acquire the sensing signals 40. Such confined holes may be understood as dedicated apertures for the sensing signals 40. Instead of a plurality of confined holes, the print bed 32 may also comprise a slit or a more complex aperture structure. The one or more evaluation location may be limited to one or more intersections, or coincidences, between the holes, slit or more complex aperture structure and the printing path.

FIG. 3 also illustrates that the controller 203 and/or the system 100 may generate a digital twin of the printed object. The digital twin may be viewed as a digital representation of the printed object, and may be determined based on sensing signals acquired throughout the printing process. It will be understood that such a digital twin may also be generated by the apparatus 200 of FIG. 2. Digital twins of printed objects may be used for a global quality assessment of printed objects, such as by comparing the digital twins to printing models based on which a respective printing process is planned. They may also be used as a basis for planning future printing processes.

How the relative movement between a) the printing device and b) the print bed and/or previously printed material, as well as between c) the printing device and d) the sensing device is realized is generally not limited to a particular way. In particular, the x-y-z Cartesion-types of movements between a) the printing device and b) the print bed and/or previously printed material which have been outlined above with respect to FIG. 2 are only exemplary. Among the many other possible configurations for achieving relative movements between a) the printing device and b) the print bed and/or previously printed material are, for instance, configurations in which the movements of a) the printing device and/or b) the print bed are described in terms of other Cartesian, polar or yet other coordinates. Possible alternative moving means may include, for instance, one or more robotic arms. Exemplary alternative movement systems include those known as CoreXY, delta, SCARA, belt and H-bot. For a given implementation of the relative movements between a) the printing device and b) the print bed and/or previously printed material, generally also a plurality of different possible implementations for the relative movements between c) the printing device and d) the sensing device will exist.

Also regarding the printing technique used, particularly the printing device 201 and the printing material 20, as well as regarding the type of sensing employed, particularly the sensing device 202, many variations are possible, with no or only little changes regarding how the printing device 201 and the sensing device 202 are controlled to move. The functions of the system 100, particularly the printed material property to be sensed, the evaluation location, how the mismatch between the sensed material property and the printed material target property is determined and/or how the control parameter is determined based on the determined mismatch, may be chosen depending on the printing technique and the sensing technique employed.

Preferably, the printing process is a bioprinting process. Hence, the apparatus 200 may be adapted for bioprinting. The bioprinting process may involve using the printing device 201 for depositing bio ink and bio material inks as printed material 20. In FIGS. 2 and 3, an extruder-type printing device 201 is shown, i.e. a device by which printed material can be extruded onto the respective print bed. However, additionally or alternatively to an extrusion-based printing process, an inkjet-based, a laser-assisted, a stereolithographic, a two-photon polymerization-based, a melt-electrospinning writing-based, an electrohydrodynamically-based or microfluidic-based printing process would be possible. Also a combination of the aforementioned types of printing processes would be possible. In principle, all of these types of printing techniques could be used for bioprinting, in particular. FIGS. 2 and 3 just serve to exemplarily and schematically illustrate an evaluation and/or control of a three-dimensional printing process, without limiting the control to any of the specific elements shown. In particular, a same or similar evaluation and/or control could be adopted when using printing devices which are not of the extruder type, and/or when the relative movements between the printing device and the print bed, or printed material, are effected differently.

The sensing signal can particularly be an imaging signal. Accordingly, the sensing device 202 shown in FIG. 2 is illustrated as an imaging device, which in this case has a generally cylindrical form with a lens portion projecting outward in a lower part of the imaging device. The types of sensing signals acquired by the imaging device which are thereafter provided for further processing to the system 100, i.e. the types of imaging devices which can be used, are essentially non-limited. For instance, an imaging device selected from any of the following may be used for acquiring the imaging signals: an OCT system, a three-dimensional line scanner, an ultrasound imaging device, a multiphoton microscope, a second harmonic imaging microscope, a confocal microscope, a photoacoustic microscope, a hyperspectral camera, a Raman-spectrometric imaging device, a Lidar scanner. In fact, any microscope or camera may be used. Also combinations of the aforementioned types of imaging devices may be used. The type of imaging device used may be chosen depending on known imaging characteristics of the respective type of imaging device, particularly depending on a degree of suitability of the respective type of imaging device for imaging the one or more printed material properties of interest. This may also depend on the type of printing material used. Nevertheless, it may not be necessary that any actual image determined based on the imaging signal is used. Instead, it may be sufficient, and therefore more time-efficient, to use a raw or only partially processed signal, i.e. an imaging signal being not yet an image, of an imaging device as an input for the mismatch determining unit. For instance, OCT spectrogram data instead of full OCT images may be acquired from the respective evaluation locations using the sensing device 202, wherein mismatches between printed material properties and printed material target properties may be determined by the mismatch determining unit 102 in the OCT spectrogram data, and wherein a respective control parameter may be determined by the control parameter determining unit 103 based on the mismatches determined in the OCT spectrogram data.

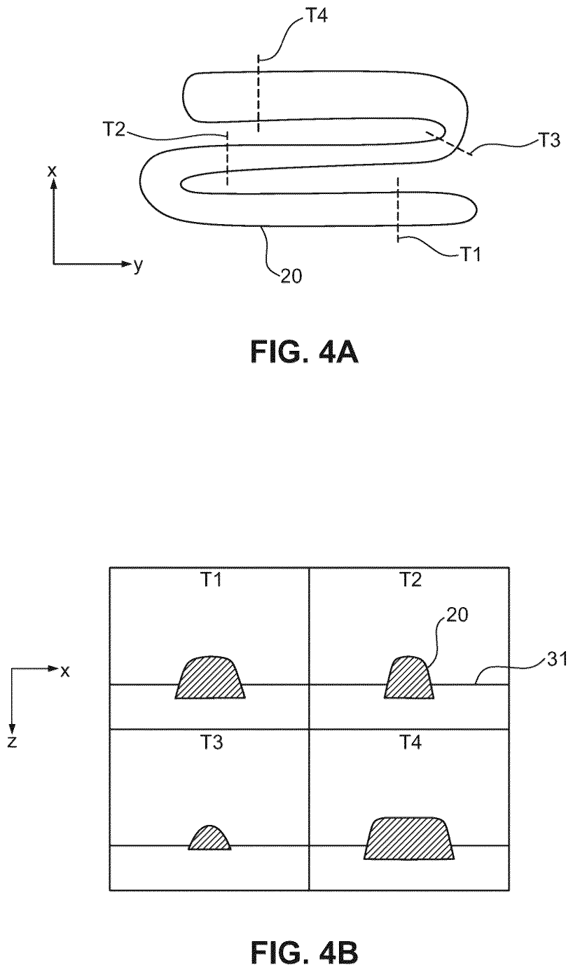

FIG. 4A shows schematically and exemplarily a strand of printed material 20, like the one shown in FIG. 2, as viewed from above, i.e. in a z direction along which the printing material could be extruded. The strand of printed material 20 shown in FIG. 4A appears two-dimensional, but it will be understood that, since the printing material 20 is deposited with some non-zero thickness, the strand of printed material 20 is three-dimensional in reality. In FIG. 4A, four printing locations along the strand of printed material 20 are indicated by dashed lines transverse to the printing direction at the respective printing locations. The four indicated printing locations are referred to by T1, T2, T3 and T4, respectively, since they are associated with respective time points during the printing process, the time points indicating when a current printing location coincided with the respective indicated printing location T1, T2, T3 and T4.

FIG. 4B shows schematically and exemplarily four slice images corresponding to two-dimensional images of cross sections through the strand of printed material 20 at the respective printing locations T1, T2, T3 and T4. Apart from the printed material 20, the print bed 31 can be seen in the images. Form FIGS. 4A and 4B, variations in a width with which the printing material 20 has been deposited onto the print bed 31 can be recognized. Such width variations may be undesirable. By use of slice images as shown in FIG. 4B and adapting suitable control parameters depending on a width of the printed material 20 identified in the images, width variations can be controlled during printing. For instance, for a predefined section of a strand of material 20 to be printed, a target width may be predefined, wherein the mismatch determining unit 102 may be configured to determine a difference between an actual width of printed material 20 and the target width for each of a plurality of evaluation locations along the predefined section of the strand of the printed material 20, based on respective slice images acquired at the evaluation locations. If the evaluation locations correspond to the respective current printing locations, for instance, the differences between the width of a currently printed portion of printing material and the target width can be determined based on, particularly in, images composed from imaging signals acquired from the respective current printing locations.

Imaging the printed material 20 allows to obtain rich information about properties of the printed material 20, which can then be compared to printed target properties. However, additionally or alternatively, other sensing techniques may be used. For instance, particularly in extrusion-based printing processes, a weight sensor can be used as sensing device in order to weigh an amount of deposited printing material 20. Such a weight sensor may, for instance, be placed below the print bed 31. Also a pressure and/or a force sensor for sensing a pressure inside an extruder used for printing may be used as sensing device, and/or a force sensor for sensing a force with which printing material 20 is extruded. Sensing signals of any of such dedicated sensors may be used and provided for processing inside the system 100, such as via the control device comprising the controller 203. Additionally or alternatively, a feedback from one of the motors used for moving the printing device 201 and/or depositing material using the printing device 201 may be used as an input to the system 100. For instance, a signal indicative of a current energy consumption of a motor used for extruding the printing material 20 may be provided by the sensing signal providing unit 101. Additionally or alternatively, a feedback from an encoder may be provided as an input to the mismatch determining unit 102, wherein mismatches based on which control parameters are determined may then refer to this feedback as compared to a target feedback. The encoder whose feedback is used may be an encoder used by the controller 203 for controlling the printing device 201.

Irrespective of how the printed material target property is determined, particularly of which sensing signal and which sensing device are used, the printing material target property may be determined based on a printing model, i.e. a model of the respective object to be printed. If, for instance, the printing model did correspond to a volumetric image of the object to be printed, for instance, a section through the volumetric image could be taken to determine slice images like those shown in FIG. 4B, wherein based on these sections target widths of printed material could be determined. However, it may be preferred that the model corresponds to a data structure defining a planned printing path and (further) control parameters along the planned printing path. The planned printing path may be given in terms of movements to be carried out by the printing device 201 in order to follow the planned printing path. The control parameters defined along the printing path may comprise a pressure or force with which printing material is to be extruded in the case of extrusion-based printing, for instance. The printed material target property may refer, for instance, to a printed material location, direction or volume determined based on the printing model. The printing model may be used to control the printing, i.e. may be used by the controller 203. Moreover, the printing model may, apart from information used for controlling the printing process, from which already some printed material target properties may be inferred, comprise further information indicative of printed material target properties. Such further information may not be used for controlling the printing process, or only insofar as it is for evaluation and a result of the evaluation is used to determine control parameters by the control parameter determining unit 103. In such a case, the printing model may be understood as a printing control and evaluation model. For instance, the printing model may define, besides the planned printing path and extrusion pressures or forces along the path, a target maximum porosity of the printed material 20. The porosity of the printed material 20, i.e. the extruded material deposited on the print bed 31, would be difficult to derive from control parameters like the extrusion pressure or force alone.

The printing model may be determined based on one or more previous printing processes. For instance, the printing model for a printing process to be carried out, i.e. for an object to be printed, may be determined based on the one or more printing models based on which one or more previous printing processes have been carried out. Additionally or alternatively, the printing model may be determined based on printing results from the one or more previous printing processes, such as based on the respective printed objects. Also a quality assessment of the printing result of the one of more previous printing processes can be taken into account for determining the printing model for a printing process to come. The printing model may be predefined, i.e. fixed, before the printing process is started. Also the printed material target property may be predefined. Alternatively, the printed material target property may be determined based on the printing model during the printing process. For instance, the mismatch determining unit 102 may be configured to retrieve the printing model from a storage and determine the printed material target property based on the retrieved printing model. While the printing material target property may be determined based on a single printing model, wherein the single printing model may itself be determined based on one or more previous printing processes, the printed material target property may also be determined based on one or more printing models, wherein the one or more printing models may be determined based on one or more previous printing processes.

The mismatch between the printed material property and the printed material target property which is determined by the mismatch determining unit 102 for a respective current printing location 10 may depend on a predefined tolerance. The tolerance predefined for this purpose may also be considered a mismatch tolerance. The mismatch tolerance may be predefined such that, for instance, mismatches between the printed material property and the printed material target property by a few percent, such up to 5 percent, preferably up to 3 or even 1 percent, for instance, are tolerated. Tolerated mismatches may cause the control parameter determining unit 103 to not adapt any control parameter. Different mismatch tolerances may be predefined for different printed material properties and/or for different evaluation locations.

The control parameter determined and/or adapted by the control parameter determining unit 103 can, in principle, be any control parameter relevant in a respective printing process. It may be preferred, however, that only those control parameters are considered for determination and/or adaption based on a mismatch for a given printed material property which are known to be correlated with the given printed material property, i.e, particularly can potentially be used to change the printed material property. Exemplary control parameters determined by the control parameter determining unit 103 include, in case the printing process is extrusion-based, a material extrusion speed, a movement speed of an extruder used as printing device 201, a pneumatic pressure, a temperature, a retraction speed and/or length of the extruder of the, or corresponding to, the printing device 201, a distance of the extruder to the print bed 31 and/or a temperature of the print bed 31, and, in case the printing process is injection-based, a droplet size, an ejection speed, and/or an ejection frequency. Further control parameters associated with various printing techniques have already been indicated above, particularly in above Table 1.

It should be emphasized that the current printing location 10, the evaluation location and a printing location to which the determined control parameter relates, i.e. at which, for instance, a change in a printing parameter is to become effective, can refer to three different locations. Moreover, for a given current printing location 10 at some point in time during the printing process, several evaluation locations may be considered. That is to say, sensing signals indicative of printed material properties at several evaluation locations may be acquired and/or provided, wherein the several evaluation locations will typically be printing locations preceding the current printing location 10 along the printing path. Likewise, the control parameter may be determined and/or adapted such that changes in the printed material property become effective at several future locations along the printing path. In fact, also more than one current printing location may, in principle, be possible. For instance, a printing device 201 may be used which deposits printing material 20 at several locations, or several printing devices 201 may be used, each depositing printing material 20 at a respective location. FIGS. 4A and 4B illustrate the exemplary case that sensing signals are acquired and/or provided for each of a plurality of printing locations T1, T2, T3 and T4 of an ongoing three-dimensional printing process, wherein the sensing signals are indicative of a printed material property like, for instance, the printed material's width at the respective printing location T1, T2, T3 and T4. The sensing signals may have been acquired from the respective printing locations T1, T2, T3 and T4 when the current printing location 10 corresponded to the respective locations T1, T2, T3 and T4, or may have been acquired later, i.e. when the current printing location 10 had already passed the respective locations T1, T2, T3 and T4. As was described with reference to FIGS. 4A and 4B, mismatches like width differences may be determined between the printed material property and the printed target property for the plurality of printing locations T1, T2, T3 and T4 based on the respective sensing signals, i.e. based on the sensing signals from the respective printing locations T1, T2, T3 and T4. Based on a respective mismatch at a given one of the printing locations T1, T2, T3 and T4, a control parameter like, for instance, an extrusion pressure or force may be adjusted for a respective subsequent printing location. The subsequent printing location, which refers to the location at which the adjustment in printing parameter is to become effective, may be chosen so as to follow the respective evaluation location T1, T2, T3 and T4 as closely as possible, or may be chosen so as to correspond to a respective following of the four evaluation locations T1, T2, T3 and T4, or in any other way.

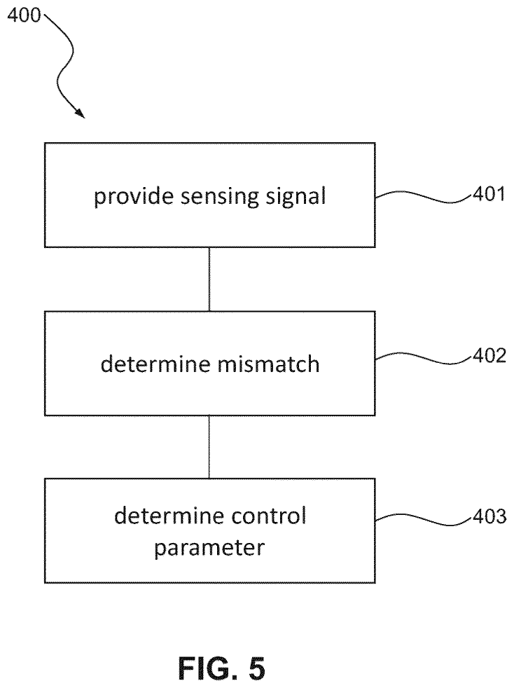

FIG. 5 shows schematically and exemplary a method 400 for controlling a three-dimensional printing process. The method includes a step 401 of providing a sensing signal 40 indicative of a printed material property at an evaluation location, the evaluation location being a printing location satisfying a predefined relationship with respect to a current printing location 10. Furthermore, the method 400 includes a step 402 of determining a mismatch between the printed material property and a printed material target property for the evaluation location, or an indicator of such a mismatch, based on the sensing signal. In a further step 403, a control parameter for the three-dimensional printing process is determined based on the determined mismatch or its indicator. It may be preferred that the predefined relationship between the evaluation location and the current printing location 10 is such that the evaluation location corresponds to the current printing location 10. However, as was discussed at length above, this does not have to be the case.

Although the above embodiments have been described with reference to a system for controlling a three-dimensional printing process, instead of a system also an apparatus or other device could be used. Moreover, the system is not necessarily a system for, i.e. suitable for, controlling a three-dimensional printing process. In particular, the suitability of the system for the purpose of controlling a three-dimensional printing process is not necessarily to be understood in the sense that the system is suitable for this purpose, particularly not by itself. Instead, it may be necessary that the system interacts with, or is used by a user in combination with, further units, devices, etc. Besides, the printing process is not necessarily three-dimensional, but can also be, for instance, one- or two-dimensional. If considering time as a dimension, the printing process can also be considered four-dimensional.

The sensing signal providing unit is dispensable, i.e. is not necessarily part of the system, which could be a system for controlling a three-dimensional printing process. For instance, the sensing signal providing unit could be replaced by a different unit or device. However, it could also be abandoned without being replaced. Moreover, although the sensing signal providing unit was described above as being configured to provide a sensing signal indicative of a printed material property at an evaluation location, the evaluation location being a printing location satisfying a predefined relationship with respect to a current printing location, this does not have to be the case.

In particular, the sensing signal providing unit is not necessarily configured to provide a sensing signal indicative of a printed material property at an evaluation location being a printing location. Instead, other data may be provided as an initial input for controlling the printing process, whether it be sensing data or not. In particular, the sensing signal providing unit, which could also be a measurement signal providing unit or device, could be configured to provide sensing or measurement signals, respectively, from non-printing locations and/or signals or other data which are not indicative of a printed material property. For instance, the sensing signal providing unit may be configured to provide a sensing signal indicative of a property of printing material before being printed, such as, for instance, from a location inside a printing device used for printing. Exemplary properties of this kind would be a viscosity, a cell density, a cell viability of the printing material inside the printing device, and/or changes in any of these. Knowing, for example, such properties of the printing material inside a printhead before the material is deposited on a print bed or previously deposited material allows to adjust printing parameters based thereon before the material leaves the printhead.

Hence, the one or more evaluation locations are not limited to printing locations, but could also be, for instance, locations inside a device. Also, whether or not the one or more evaluation locations are printing locations, they do not necessarily satisfy a predefined relationship with respect to the current printing location. For instance, the one or more evaluation locations may be determined based on a user input, e.g. a user input received by the system via a user interface, and/or may be determined during the printing process, particularly in a manner that could not be captured in terms of a predefined relationship with respect to a respective current printing location. This could be the case, for instance, if the one or more evaluation locations were determined at least partially randomly. Furthermore, instead of a current printing location, the evaluation location could be predefined or otherwise determined with respect to any other location.

The sensing signal providing unit can be a receiving unit configured to receive one or more sensing signals from, for instance, the sensing device and to provide the received one or more sensing signals. However, the sensing signal providing unit can also be or comprise a storage in which one or more previously acquired sensing signals have been stored and from which they can be retrieved for providing the same. The sensing signal providing unit can also be or comprise the sensing device. Optionally, the sensing signal providing unit can be configured to pre-process the one or more sensing signals before providing them, i.e. for further processing.

Also the planned printing path data providing unit can be or comprise a receiving unit for receiving the planned printing path data, a storage for storing the planned printing data for access and/or a processing unit for determining the planned printing path data from previously received and/or stored data.

The mismatch determining unit is dispensable, too, i.e. also this unit is not necessarily part of the system, which could be a system for controlling a three-dimensional printing process. For instance, the mismatch determining unit could be replaced by a different unit or device. However, it could also be abandoned without being replaced. Moreover, although the mismatch determining unit was described above as being configured to determine a mismatch between the printed material property and a printed material target property for the evaluation location based on the sensing signal, this does not have to be the case. For instance, the mismatch determining unit, which could be regarded as a data processing unit, could be configured to determine another quantity than a mismatch, wherein, if desired, also such a different quantity could be taken as a basis for determining or adapting a control parameter. Moreover, the quantity determined, i.e. the mismatch or other quantity, is not necessarily determined based on any sensing signal, and also not necessarily based on any of the printed material property and the printed material target property for the evaluation location. Hence, in general, for controlling the printing process, any data may be provided, wherein this data may be used to determine a control quantity. The control quantity may also be provided directly.

For instance, additionally or alternatively to any sensing signals and/or mismatches determined based thereon, control parameters may be determined based on planned printing path data and, optionally, a current printing location. If, for instance, it is determined that the current printing location approaches a curve in the planned printing path and if curves in printing paths are associated with inaccuracies, control parameters for the curve can be adjusted in advance. An association between segments like curves in printing paths and printing inaccuracies may be established based on previous printing processes. An exemplary adjustment in control parameters for a curve being approached by a current printing location could be such that, such as by means of an increased extrusion pressure or force, more printing material is being deposited along the curve as compared to printing path segments before and/or after the curve, and/or as compared to straight printing path segments.

In a further variant, the control parameter determination unit may be configured to use a trained machine learning unit to determine the control parameters for the three-dimensional printing process. The machine learning unit may have been trained based on previous printing processes. An input of the machine learning unit may comprise sensing signals and/or other data as indicated above, and an output of the machine learning unit may comprise the control parameters. The training of the machine learning unit may involve an evaluation of local and/or global printing results during the previous printing processes. Such an evaluation may include a determination of mismatches between printed material properties and printed material target properties, but may additionally or alternatively include other types of evaluations. For example, the machine learning unit may be configured to model the printing process. The model may be a microscopic model of material deposition depending on control parameters like an extrusion pressure or force, the respective printing material, and a geometry of an extruder used as printing device. Based on the model, expected printing results may be determined for several control parameter values, wherein the expected printing results may be evaluated based on a predefined evaluation measure. Based on current control parameters in a printing process, which may be estimated based on, potentially sensed, current and/or previous printing characteristics, and based on the model of the printing process, the machine learning unit may provide adjusted control parameters as output. While using a trained machine learning unit for determining control parameters can lead to a more accurate control of a printing process, this may also increases the complexity of the control, and therefore potentially also the time needed for determining, or adjusting, control parameters. This may limit a printing speed. Hence, it may be preferred to not use a machine learning unit.

Also the control parameter determining unit is dispensable, i.e. not necessarily part of the system, which could be a system for controlling a three-dimensional printing process. For instance, the control parameter determining unit could be replaced by a different unit or device. However, it could also be abandoned without being replaced. Moreover, although the control parameter determining unit was described above as being configured to determine a control parameter for the three-dimensional printing process based on the determined mismatch, this does not have to be the case. For instance, the control parameter determining unit, which could be regarded as a data processing unit which is separate from or combined with the mismatch determining unit, could be configured to determine the control parameter directly based on the sensing signal or other provided data, or may be configured to directly output control signals for the three-dimensional printing process which cannot be interpreted as corresponding to a control parameter.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfil the functions of several items recited in the claims. The mere fact that certain measures are recited in mutually different dependent claims does not indicate that a combination of these measures cannot be used to advantage. Procedures like the providing of a sensing signal, the determining of a mismatch, the determining of a control parameter, et cetera, performed by one or several units or devices can be performed by any other number of units or devices. These procedures can be implemented as program code means of a computer program and/or as dedicated hardware. A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a system for controlling a three-dimensional printing process. The system comprises a sensing signal providing unit configured to provide a sensing signal indicative of a printed material property at an evaluation location. The evaluation location is a printing location satisfying a predefined relationship with respect to a current printing location and can, in particular, correspond to the current printing location. The presented system further comprises a mismatch determining unit configured to determine a mismatch between the printed material property and a printed material target property for the evaluation location based on the sensing signal, and a control parameter determining unit configured to determine a control parameter for the three-dimensional printing process based on the determined mismatch. The system allows for an increased reproducibility of three-dimensional printing results.

1. A system (100) for controlling a three-dimensional printing process, comprising:
a sensing signal providing unit (101) configured to provide a sensing signal indicative of a printed material property at an evaluation location, the evaluation location being a printing location satisfying a predefined relationship with respect to a current printing location (10),
a mismatch determining unit (102) configured to determine a mismatch between the printed material property and a printed material target property for the evaluation location based on the sensing signal, and
a control parameter determining unit (103) configured to determine a control parameter for the three-dimensional printing process based on the determined mismatch,
wherein the evaluation location corresponds to the current printing location.

2. The system (100) according to claim 1, wherein the printing process is a bioprinting process.

3. The system (100) according to claim 1, wherein the printing process is any one or any combination of the following: extrusion-based, inkjet-based, laser-assisted, stereo-lithographic, based on two-photon polymerization, based on melt-electrospinning writing, based on electrohydrodynamic jetting, based on microfluidic dispensing.

4. The system (100) according to claim 1, wherein the printed material property refers to any of the following: a location or alignment of the printed material, particularly with respect to printed material at a previous printing location, a shape parameter, a surface area, a porosity, a cellular density, a material and/or cell percentage, a material type distribution, a pore size, a strut size, a pore shape parameter, a pore interconnectivity, a degree of fusion between layers, a weight, a degree of humidity, a degree of cross-linking.

5. The system (100) according to claim 1, wherein the sensing signal is an imaging signal.

6. The system (100) according to claim 5, wherein the imaging signal is any of the following: an optical coherence tomography signal, a three-dimensional line scanner signal, an ultrasound signal, a multiphoton microscopy signal, a second harmonic generation signal, a confocal microscopy signal, a photoacoustic microscopy signal, a hyperspectral imaging signal, a Raman spectroscopy signal, a Lidar signal.

7. The system (100) according to claim 5, wherein the mismatch determining unit (102) is configured to determine the mismatch between the printed material property and the printed material target property for the evaluation location based on an image determined based on the imaging signal.

8. The system (100) according to claim 1, wherein the sensing signal is any of the following: a signal of a weight sensor for weighing an amount of deposited material, a signal of a pressure sensor for sensing a pressure inside an extruder used for printing, a signal of a force sensor for sensing a force with which printing material is extruded in case of extrusion-based printing, a signal indicative of a current energy consumption of a motor used for extruding the printing material.

9. The system (100) according to claim 1, wherein the printed material target property is determined based on a printing model, wherein the printing model is a model of an object to be printed.

10. The system (100) according to claim 9, wherein the printing model is determined based on one or more previous printing processes.

11. The system (100) according to claim 1, wherein the mismatch determining unit (102) is configured to determine the mismatch between the printed material property and the printed material target property for the current printing location (10) based further on a predefined tolerance.

12. The system (100) according to claim 1, wherein the control parameter refers to any of the following:
in case the printing process is extrusion-based: a material extrusion speed, a movement speed of an extruder used as printing device (201), a pneumatic pressure, a temperature, a retraction speed and/or length of the extruder, a distance of the extruder to a print bed (31) and/or a temperature of the print bed (31),
in case the printing process is inkjet-based: a droplet size, an ejection speed, and/or an ejection frequency.

13. The system (100) according to claim 1, wherein:
the sensing signal providing unit (101) is configured to provide sensing signals for each of a plurality of printing locations (T1-T4) of an ongoing three-dimensional printing process, the sensing signals being indicative of the printed material property at a respective one of the plurality of printing locations (T1-T4),
the mismatch determining unit (102) is configured to determine mismatches between the printed material property and the printed material target property for the plurality of printing locations (T1-T4) based on the respective sensing signals, and
the control parameter determining unit (103) is configured to determine, based on a respective mismatch at a given one of the plurality of printing locations (T1-T4), the control parameter for a respective subsequent printing location.

14. An apparatus (200) for three-dimensional printing, comprising:
a printing device (201) for printing material in three dimensions,
a sensing device (202) for acquiring sensing signals indicative of a printed material property at a current printing location (10), and
a controller (203) for controlling the printing device (201) based on a control parameter determined by the system (100) according to using the sensing signals acquired by the sensing device (202).

15. The apparatus (200) according to claim 14, wherein the printing device (201) and the sensing device (202) are movable relative to each other.

16. The apparatus (200) according to claim 14, wherein the sensing device (202) is movable so as to follow the printing device (201) in a printing direction.

17. The apparatus (200) according to claim 14, wherein the sensing device (202) is configured such that the sensing signals are acquired from the current printing location (10) and from a region within a predefined distance from the current printing location (10).

18. The apparatus (200) according to claim 14, wherein the sensing device (202) is rotatable relative to the printing device (201).

19. A method (400) for controlling a three-dimensional printing process, including:
providing (401) a sensing signal indicative of a printed material property at an evaluation location, the evaluation location being a printing location satisfying a predefined relationship with respect to a current printing location (10),
determining (402) a mismatch between the printed material property and a printed material target property for the evaluation location based on the sensing signal, and
determining (403) a control parameter for the three-dimensional printing process based on the determined mismatch,
wherein the evaluation location corresponds to the current printing location.

20. A computer program for controlling a three-dimensional printing process, comprising instructions causing a computer to carry out the method (400) according to claim 19.
